(19) United States
(12) Patent Application Publication
(10) Pub. No.: US 2009/0202597 A1
(43) Pub. Date: Aug. 13, 2009

(54) Ache-Nmda Combination Wafer

(76) Inventors: Hans-Rainer Hoffmann, Neuwied (DE); Reto Brandli, San Fransisco, CA (US); Frank Theobald, Wehr (DE)

Correspondence Address: D Peter Hochberg;D Peter Hochberg Co, 1940 East 6th St, Cleveland, OH, 44114, US

(21) Appl. No.: 12/308,236

(22) PCT Filed: Jun. 4, 2007
(86) PCT No.: PCT/EP2007/004951

§ 371 Date: Jan. 22, 2009

(30) Foreign Application Priority Data
Jun. 16, 2006 (DE) 10 2006 027 791.0

(51) Int. Cl. A61K 9/00; A61K 31/13

(52) U.S. Cl. 424/400; 514/662

(57) ABSTRACT

Sheet-like pharmaceutical preparations based on hydrophilic polymers, which rapidly disintegrate upon contact with moisture and which are used to treat dementia-related illnesses The dosage form contains an active agent combination of at least two active agents which are suitable for the treatment of dementia (antidementia agents). The antidementia agents should be chosen from the group comprising acetyl cholinesterase inhibitors (AChE inhibitors) and NMDA antagonists (n-methyl-D-asparaginic acid antagonists). The use of such an active agent combination for producing an orally administrable pharmaceutical product for the treatment of dementia-related illnesses such as Alzheimer's disease, as well as a procedure for the symptomatic treatment of Alzheimer's disease by the oral administration of one of the above pharmaceutical products is also provided.

### CROSS-REFERENCE TO RELATED APPLICATIONS

This application is a National Stage application of International Application No. PCT/EP2007/004951, filed on Jun. 4, 2007, which claims priority of German application number 10 2006 027 791.0, filed on Jun. 16, 2006, both of which are incorporated herein by reference in their entireties.

### BACKGROUND OF THE INVENTION

1. Field of the Invention

The present invention relates to a sheet-like pharmaceutical preparation based on hydrophilic polymers, which rapidly disintegrates upon contact with moisture and which is used to treat dementia-related illnesses, wherein the dosage form contains an active agent combination of at least two active agents which are suitable for the treatment of dementias (antidementia agents).

The invention further relates to the use of such an active agent combination for the production of an orally administrable pharmaceutical product for the treatment of dementia-related illnesses such as Alzheimer's disease, as well as to a method for the symptomatic treatment of Alzheimer's disease by means of the oral administration of one of the above pharmaceutical products.

2. Description of the Prior Art

In Germany at present around 1.2 million people suffer from dementia—with an upwards trend. The reason for this is to seen in the rising number of people of advanced age and in the fact that the risk of becoming afflicted by dementia increases with age. Whereas “only” one in twenty people at an age between 65 and 69 suffers from dementia, among people aged between 80 and 90 the proportion of afflicted persons is as high as almost one third.

As the proportion of senior citizens in the total population is expected to rise, we will have to expect an increase in dementia cases as well. Experts anticipate a doubling of the number of cases by the year 2030.

Alzheimer's is presently the most common form of dementia, with dementia being understood as the loss of cognitive functions such as thinking, memorizing and linking of thought contents, to an extent that activities and sequences of activities in everyday life can no longer be carried out independently. In its final stage, dementia can also be fatal.

Alzheimer's disease is defined in the literature as a progressive, degenerative illness of the CNS which is associated with impairments of memory, of intelligence and of behaviour; the loss of memory may affect not only personal recollections but also actions as elementary as ingestion of food, spatial orientation, basic vocabulary, and the like. One of the causes of this impairment of memory performance is an impairment of the neurotransmitters glutamate and acetylcholine.

With the increasing average age of the population, Alzheimer's disease (Morbus Alzheimer), or Alzheimer for short, as with all dementia-related illnesses, produces a rising number of patients requiring treatment.

Since the illness is a degenerative, progressive illness without any prospect of a cure, the aim of the treatment must be to achieve an improvement, or at least a stabilization and delay of the decline in mental capacity. This not only requires non-medicinal measures such as memory training, but also an appropriate medicinal therapy.

In advanced dementia, the therapy ultimately focuses on maintaining the patient's ability to perform activities of daily living and on delaying care dependency and referral to a nursing home.

As explained above, there are presently no effective agents available to heal Alzheimer, nevertheless there are a number of medicaments that may slow down the progression of the illness and bring about an improvement of the symptoms.

There are, in particular, two groups of pharmaceuticals available for the medicinal treatment of Alzheimer dementia that have a positive influence on the messenger substances glutamate and acetylcholine, which are impaired in dementias.

One of these groups are the neuroprotective NMDA antagonists (N-methyl-D-asparaginic acid), including, for example, memantine.

Memantine stops the uptake of glutamate at the NMDA receptors so that the permanent stimulus overload is reduced and in the conduction of the stimuli the signals can be recognised again. The death of the nerve cells as a result of continuous overload can in this way be prevented or delayed. The patients become more mentally active again and their ability to perform activities of daily living increases. Visible improvements can also be detected in patients who are already in need of nursing care.

A further group of pharmaceuticals for symptomatic treatment of Alzheimer are the ACHE inhibitors (acetylcholinesterase inhibitors), which prevent the decomposition of the neurotransmitter acetylcholinesterase (ACHE) and thereby have a positive influence on signal conduction in the brain.

In addition to the above-mentioned advantageous effects on the patient, one reason why a sufficient medicinal therapy is of particular importance is that it can drastically reduce the time spent in caregiving and thereby reduce the costs of care. It is thereby possible not only to save costs but also to gain time required for the non-medicinal therapy.

However, in the therapy of Alzheimer's disease in particular, and in the therapy of dementia in general, the following problems are frequently faced:

Due to the patient's lack of memory skills the required compliance is missing, that is, taking the medicaments is frequently forgotten, which in turn reduces the memory performance still further, which in turn leads to a further decrease in taking the necessary medicaments. In extreme cases, the patients “unlearn” to swallow oral dosage forms, so that these patients are no longer accessible to oral forms of therapy.

In oral therapy, checking whether the patient has in fact taken the medication (tablet not swallowed, forgotten to swallow, etc.) often presents problems.

Furthermore, the therapy is made more difficult by the fact that not only one medicament needs to be taken but frequently a combination of different medicaments is used. Each additional medicament, however, increases the risk of forgetting or omitting to take this medicament as well as, frequently, other required medicaments which the patients, who are as a rule elderly, need to take. Moreover, along with the rising number of the tablets that need to be taken, the reluctance to take them in many cases increases also, so that the patient's compliance declines.

The task underlying the invention was therefore to provide pharmaceutical preparations by means of which it is possible to carry out a combination therapy for the symptomatic treatment of Alzheimer in a simple and safe manner and by means of which it is possible to prevent or reduce the above mentioned disadvantages. A further task underlying the invention was to indicate methods for the medicinal combination therapy of dementia-related illnesses.

Dosage forms commonly utilised for administration of active agents in therapy are tablets or capsules.

Tablets or capsules are easy to take, but they make it more difficult to check whether they have actually been taken, and the active agents, when absorbed via the gastrointestinal tract, are subject to the “first-pass effect”, thus necessitating high initial active agent concentrations in the tablet or capsule in order to achieve the desired therapeutic effects.

Furthermore, it is known to use buccal or sublingual tablets that release the active agent in the oral cavity so that it can be absorbed directly via the oral mucosa.

The disadvantage of such tablets is an often unpleasant mouthfeel and, on account of their compact form, an only slow disintegration of the tablets, and, as a consequence thereof, a slow release of the active agents. Moreover, in patients who have unlearned how to swallow or who are highly confused there is a risk of “choking” when the tablets or capsules are being administered.

### SUMMARY OF THE PRESENT INVENTION

The task of the present invention is solved in that at least two active agents that belong to the group of the antidementia agents are contained in a hydrophilic polymer film which quickly disintegrates after application in the oral cavity. Preferably, one active agent contained in the hydrophilic polymer film is an acetylcholinesterase inhibitor (ACHE inhibitor) and the second active agent is an NMDA antagonist (N-methyl-D-asparaginic acid antagonist).

The improved therapy success of an active agent combination in the treatment of dementias is due to the fact that different active agents are effective via different mechanisms of action, whereby the positive effects on memory skills complement or potentiate each other.

The doses of the individual active agents may thus be lowered or their effect enhanced, as the case may be.

Even where active agents are combined, a consistent intake and good compliance of the medicament are a prerequisite to ensuring optimal efficacy.

The administration of these active agent combinations in sheet-like dosage forms (wafers) not only enables easy intake but also the exact adaptation of the active agent components to each another, so that false dosages because intake has been forgotten or because of double intake of only one active agent, which is a problem particularly in dementia patients, and consequently an insufficient therapy, do not occur.

By means of the orally applicable dosage form in the form of a wafer made of a quickly disintegrating hydrophilic polymer, intake of the medicaments by the patient is guaranteed since the wafer immediately disintegrates in the mouth, so that the administration and supervision of intake is made easier for the nursing staff.

In addition, the dosage form of the invention already contains an active agent combination adapted to the therapy, so that the administration of the medicaments to dependent patients needs to be supervised only once.

For patients who are themselves responsible for their medication, the wafer comprising an active agent combination considerably facilitates intake, so that the necessary consistent intake according to an intake schedule is guaranteed. Both compliance and therapy success are improved, and the risk of false applications is minimised.

The absorption of the active agents via the oral mucosa, as compared to other peroral dosage forms, affords the additional advantages that also patients having difficulty swallowing, patients refusing to take tablets, or patients who because of dementia have unlearned to swallow can be administered medicaments via the oral route.

In addition, as a result of the parenteral administration, i.e. the direct absorption of the active agent via the mucous membrane, the active agents are not subject to the first-pass effect. Since in this way no active agent is metabolised before it reaches its site of action, the initial dose can be kept as low as possible. Furthermore, fluctuations in active agent concentration due to incomplete or delayed absorption, and delayed onset of action depending on the previously ingested quantity of food are suppressed or minimised. Hence, the patient can be stabilised more reliably to the required treatment dose, and the necessity of taking the medicament on an empty stomach, for example, can be dropped.

Furthermore, a single active agent combination may contain active agents with different mechanisms of action having a synergistic effect, so that as a result of the different physiological activity and of the treatment of different symptoms of dementia, lower amounts of the active agents can be dosed than would be the case with single-component compositions. A positive cumulative effect of this kind is known to occur with active agent combinations of memantine with an ACHE inhibitor.

In the dosage forms according to the present invention, combinations of different AChE inhibitors, nootropics and NMDA antagonists are used, it also being possible to use several active agents of one group in the pharmaceutical product according to the invention.

The wafers may contain up to five, preferably up to three, and more preferably two, active agents, with at least one of these active agents being an ACHE inhibitor, an NMDA antagonist or a nootropic.

By varying the ratio of the active agents to each other, it is possible to adapt the dosages to the respective needs and forms of treatment. If the patient's mental condition changes, he or she can be easily stabilised on a dosage form with an altered active agent combination, which is, as a rule, a combination with higher active agent concentration.

Because of the simple and low-cost manufacture of the wafers, it is possible to provide a large number of medicaments containing different active agent concentrations.

If the wafer is made up of a laminate, it is possible, for example, to alter only the layer thickness of an active agent-containing layer, or to alter the concentration of the active agent.

On the other hand, medicaments can be produced which have different active agent contents but the same active agent ratio, simply by means of cutting the surface of the dosage form to different sizes.

Furthermore, because of their flat shape, the wafers of the invention, which contain the active agent combinations, can be carried along easily, for example in a wallet, and are available at once, even when travelling. They are easy to take and they take effect quickly, which facilitates regular intake for patient's who are still mobile.

Suitable antidementia agents for use in a combination wafer comprise the ACHE inhibitors, NMDA antagonists and nootropics.

### DETAILED DESCRIPTION OF THE PRESENT INVENTION

In a preferred embodiment, the active agent combination consists of at least two active agents, selected from the group which comprises AChE inhibitors, NMDA antagonists and nootropics.

Suitable ACHE inhibitors in this respect are rivastigmin, galanthamine and donezepil, as well as pharmaceutically acceptable salts of these active agents. As an NMDA antagonist, mentamine may be used. The group of the nootropics that are used according to the invention includes piracetam and naftidrofuryl.

In addition, the preparation may contain further active agents that are classed with the vasodilators, with the calcium antagonists and with agents stimulating the blood flow in general.

Suitable for the therapy of dementia-related illnesses are furthermore sheet-like dosage forms containing at least one active agent selected from the group which comprises nimodipine, dihydroergotoxine, piracetam, pentoxyfylline, naftidrofuryl, Ginkgo biloba extracts, as well as pharmaceutically acceptable salts of these active agents. Preferably, one of these active agents is combined with an ACHE inhibitor and/or an NMDA antagonist.

The total active agent content of the wafers is between 5% and 50%, preferably between 10% and 30%, and more preferably between 15% and 25%, relative to the total weight of the wafer.

The ratio of the active agents to each other can be varied freely and depends on the potency of the active agent and on the desired effect and the required dosage.

Suitable for producing the dosage forms are water-soluble or swellable polymers that form a hydrophilic water-soluble and/or swellable polymer film. The polymers of the dosage form matrix are selected from the group comprising dextran, polysaccharides, inclusive of starch and starch derivatives, cellulose derivatives, such as carboxymethyl cellulose, ethyl cellulose or propyl cellulose, hydroxypropylmethyl cellulose, hydroxypropyl cellulose, sodium carboxymethyl cellulose (e.g. WALOCEL®), methyl cellulose, hydroxyethyl cellulose and hydroxypropylethyl cellulose, polyvinyl alcohols, polyethylene glycols, polyacrylic acids, polyacrylates, polyvinylpyrrolidones, alginates, pectins, gelatine, alginic acid, collagen, chitosan, arabinogalactan, galactomannan, agar-agar, agarose, carrageenan natural gums, tragacanth, highly dispersed silicon dioxide, bentonite, as well as derivatives of the aforementioned hydrophilic polymers or combinations of two or more of these polymers. As an alternative, the polymer film may be made of a polyvinyl alcohol-polyethylene glycol graft copolymer.

The proportion of polymer in a dosage form according to the invention is preferably 5 to 95%-wt., more preferably 15 to 75%-wt., relative to the dry mass of the dosage form.

To improve the physicochemical properties, for example reduce the brittleness or embrittlement, humectants, such as glycerine, propylene glycol, sorbitol, mannitol, polyethylene glycol, polyglycerol ester and the like, may be added to the film.

In a further embodiment, antioxidants, for example vitamin C (ascorbic acid), ascorbyl palmitate, vitamin E (tocopherol acetate), hydroxybenzoic acid derivatives, may be added to the wafer, in order to stabilise the film and the active agents. Furthermore, acidic and basic ion exchangers may be used as stabilisers.

In further embodiments, further ingredients such as dyes, pigments, taste flavourings, natural and/or synthetic flavouring substances, sweeteners, buffering systems, may be added to the film. In particular, the taste flavourings and flavouring substances can cover the often bad inherent taste or smell of the active agents and/or give the dosage form a pleasant taste, so that the patient's readiness to take the medication is considerably improved. To mask the taste, it is also possible for the active agent(s) of the preparation to be bound to an acidic or basic ion exchanger.

The addition of buffering systems on the one hand serves to stabilise the film and the active agents against outside influences and during storage; on the other hand, the pH of the dosage form can thereby be adjusted to a physiologically acceptable pH value, so that irritation of mucous membranes is avoided. By using a buffering system, it is also possible to improve the solubility of acidic or basic active agents in the matrix.

The dosage forms according to the invention are configured so as to be thin, for example in the form of a wafer. The thickness of the dosage form is preferably 0.1 to 5 mm, more preferably 0.5 to 1 mm. The lower limit for the thickness of the dosage form is about 50 μm. The surface area of the dosage form is between 0.09 cm² and 12 cm², preferably between 1 cm² and 8 cm², and more preferably between 3 cm² and 6 cm².

In a further embodiment, the wafers of the present invention contain a disintegrant or a wicking agent, for example a bicarbonate-acid mixture or an aerosil, being activated by contact with a liquid and accelerating the disintegration of the wafer after application thereof, and thereby also accelerating the release of active agent.

In another preferred embodiment, the wafer is present as a foam so that the release of active agent takes place even more rapidly because of the enlarged surface. In this embodiment, the cavities of the foam may contain one or more of the active agents or auxiliary agents in liquid form.

To improve the absorption of the active agents via the mucous membrane, permeation enhancers, such as substances from the groups of the fatty alcohols, fatty acids, polyoxyethylene fatty alcohol ethers, polyoxyethylene fatty acid esters, fatty alcohol esters and fatty acid esters, particularly sorbitan monolaurate or esters of long-chain fatty acids with methyl, ethyl or isopropyl alcohol, or esters of fatty alcohols with acetic acid or lactic acid, or substances such as DMSO (dimethyl sulfoxide) and oleic acid diethanolamine may likewise be incorporated in the film. The constituent amount of these substances, if present, is 0.1%-wt. to 25%-wt., preferably 1 to 10%-wt., in each case relative to the total weight of the active agent matrix.

Furthermore, the composition of the wafer may contain compounds that retard the release of active agent (e.g., microencapsulation).

In a further embodiment, the wafer has mucoadhesive properties, so that it adheres to the mucous membrane until it is completely dissolved. This embodiment, in addition, makes it easier to supervise the administration of the medicaments since the wafer, which adheres to the mucous membrane, cannot be spit out.

In another preferred embodiment, at least one of the active agents is bound to an ion exchanger, so that the hydrophilic polymer disintegrates quickly in the oral cavity, whereas the release of active agent is retarded or occurs when the pH has changed, e.g. in the gastrointestinal tract. In this way, active agents having a different mechanism of action and absorption can be administered in one dosage form, that is, at least one of the released active agents is absorbed at the site of application, for example via the mucous membrane, and the other active agent is transported farther and absorbed at another site.

The wafer may also be made up as a laminate with different layers, with the active agents being contained in discrete layers which are spatially separated from each other and differ from each other in terms of their composition. In this way, the active agents can be released at different sites of action, but also with retardation if the disintegration times of the various layers of the wafer differ from each other.

Likewise, the active agents may be arranged within layers that disintegrate at different rates, so that the preparation as a whole shows a retardation effect.

In a further embodiment, only one of the outer layers may be mucoadhesive, to promote the adherence of the dosage form on the mucous membrane and to facilitate the active agent absorption via the mucous membrane by establishing direct contact.

The disintegration of the inventive dosage form in an aqueous medium preferably takes place in the range from 1 second to 5 minutes, more preferably in a range from 5 seconds to 1 minute, and most preferably in the range from 10 seconds to 30 seconds.

The dosage forms according to the invention are advantageously suitable for administering medicaments in the oral cavity or for rectal, vaginal or intranasal administration.

The present invention furthermore relates to the use of an active agent combination according to the invention for the production of an oral dosage form for treating dementia-related illnesses, said dosage form preferably being formulated as a wafer.

Furthermore, the present invention relates to a method for the therapeutic treatment of a person suffering from dementia, wherein the administration of an above-described active agent combination of antidementia agents is carried out by means of an orally applicable dosage form with at least partial transmucosal absorption of at least one active agent.

Finally, the present invention also relates to a method for the production of a sheet-like dosage form, comprising the following steps:
preparing a solution containing at least one polymer and at least two active agents from the antidementia group of drugs;
spread-coating the solution on a coating substrate, and
solidifying the spread-coated solution by drying and withdrawing the solvent.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

We claim:

1. A sheet-like pharmaceutical preparation (dosage form) based on hydrophilic polymers, which rapidly disintegrates upon contact with moisture and which is used to treat dementia-related illnesses, wherein the dosage form contains an active agent combination of at least two active agents which are suitable for the treatment of dementias, and wherein said preparation contains at least one further active agent for treating dementias which is selected from the group consisting of vasodilators, calcium antagonists and agents stimulating the blood flow.

2. The pharmaceutical preparation according to claim 1, wherein the active agents are selected from the group consisting of acetyl cholinesterase inhibitors (AChE inhibitors), NMDA antagonists (N-methyl-D-asparaginic acid antagonists) and nootropics.

3. The pharmaceutical preparation according to claim 1, wherein at least one of the active agents is an AChE inhibitor and/or at least one of the active agents is an NMDA antagonist.

4. The pharmaceutical preparation according to claim 2, wherein the AChE inhibitor is selected from the group consisting of rivastigmine, galanthamine and donezepil, as well as pharmaceutically acceptable salts of said active agents.

5. The pharmaceutical preparation according to claim 2, wherein the NMDA antagonist is memantine.

6. The pharmaceutical preparation according to claim 2, wherein the nootropic is selected from the group consisting of piracetam and naftidrofuryl.

7. The pharmaceutical preparation according to claim 1, wherein the preparation further comprises at least one active agent selected from the group which comprises nimodipine, dihydroergotoxine, pentoxyfylline, naftidrofuryl and Ginkgo biloba extracts, as well as pharmaceutically acceptable salts of said active agents.

8. The pharmaceutical preparation according to claim 1, wherein the hydrophilic polymer is selected from the group consisting of dextran, polysaccharides, inclusive of starch and starch derivatives, cellulose derivatives, polyvinyl alcohols, polyethylene glycols, polyacrylic acids, polyacrylates, polyvinylpyrrolidones, alginates, pectins, gelatine, alginic acid, collagen, chitosan, arabinogalactan, galactomannan, agar-agar, agarose, carrageenan natural gums, tragacanth, highly dispersed silicon dioxide, bentonite, as well as derivatives of the aforementioned hydrophilic polymers or combinations of two or more of said polymers.

9. The pharmaceutical preparation according to claim 1, wherein the polymer film comprises a polyvinyl alcohol-polyethylene glycol graft copolymer.

10. The pharmaceutical preparation according to claim 1, wherein the preparation further comprises a humectant selected from the group consisting of glycerine, propylene glycol, sorbitol, mannitol, polyethylene glycol and polyglycerol ester.

11. The pharmaceutical preparation according to claim 1, wherein the preparation further comprises an antioxidant selected from the group consisting of vitamin C (ascorbic acid), ascorbyl palmitate, vitamin E (tocopherol acetate), hydroxybenzoic acid derivatives.

12. The pharmaceutical preparation according to claim 1, wherein the active agent of the preparation is bound to an acidic or basic ion exchanger for taste masking.

13. The pharmaceutical preparation according to claim 1, wherein the preparation further comprises dyes and/or pigments.

14. The pharmaceutical preparation according to claim 1, wherein the preparation further comprises natural and/or synthetic flavouring substances.

15. The pharmaceutical preparation according to claim 1, wherein the preparation further comprises a disintegrant or a wicking agent.

16. The pharmaceutical preparation according to claim 1, further comprising a buffer system for adjusting the pH value of the preparation.

17. The pharmaceutical preparation according to claim 1, wherein the hydrophilic polymer disintegrates within less than 5 minutes after application in the oral cavity of a user of the pharmaceutical preparation.

18. The pharmaceutical preparation according to claim 1, wherein the hydrophilic polymer disintegrates quickly in the oral cavity whereas the active agent remains bound to an ion exchanger which releases said active agent only upon reaching the gastrointestinal tract of a user of the pharmaceutical preparation.

19. The pharmaceutical preparation according to claim 1, wherein the active agents are contained in discrete layers which are spatially separated from each other and which differ from each other in terms of their respective composition.

20. The pharmaceutical preparation according to claim 1, wherein the preparation is present as a foam having cavities and at least one of the active agents is present in liquid form within the cavities of said foam.

21. Use of an active agent combination according to claim 1, for the production of an orally administrable pharmaceutical product in the form of a wafer for treating dementia-related illnesses.

22. A method for the therapeutic treatment of a person suffering from dementia, comprising the step of orally administering to the person an active agent combination based on hydrophilic polymers, which rapidly disintegrates upon contact with moisture and which is used to treat dementia-related illnesses, wherein the dosage form contains an active agent combination of at least two active agents which are suitable for the treatment of dementias, and wherein said preparation contains at least one further active agent for treating dementias which is selected from the group consisting of vasodilators, calcium antagonists and agents stimulating the blood flow.

23. The method according to claim 22, further comprising the step of administering the active agent combination by a quickly disintegrating wafer which contains said combination.

24. The pharmaceutical preparation according to claim 8, wherein said cellulose derivatives are selected from the group consisting of carboxymethyl cellulose, ethyl cellulose or propyl cellulose, hydroxypropylmethyl cellulose, hydroxypropyl cellulose, sodium carboxymethyl cellulose, methyl cellulose, hydroxyethyl cellulose and hydroxypropylethyl cellulose.

25. The pharmaceutical preparation according to claim 17, wherein the hydrophilic polymer disintegrates within less than 3 minutes after application in the oral cavity.

26. The pharmaceutical preparation according to claim 25, wherein the hydrophilic polymer disintegrates within less than 1 minute after application in the oral cavity.

27. The pharmaceutical preparation according to claim 26, wherein the hydrophilic polymer disintegrates within less than 30 seconds after application in the oral cavity.
